SEQUENCE LISTING (18 sequences)

SEQ ID NO: 1 - DNA, 27 bp, Artificial Sequence
  Primer
tctgtccttcttcacaaacaagtttac

SEQ ID NO: 2 - DNA, 19 bp, Artificial Sequence
  Primer
ctgctctggacgctggaga

SEQ ID NO: 3 - DNA, 26 bp, Artificial Sequence
  Probe
tccactttcgcaattccaatggcttc

SEQ ID NO: 4 - DNA, 24 bp, Artificial Sequence
  Primer
tgcctgtcctccactagtattgaa

SEQ ID NO: 5 - DNA, 23 bp, Artificial Sequence
  Primer
aaatgatgggtaagtcaacgcat

SEQ ID NO: 6 - DNA, 29 bp, Artificial Sequence
  Probe
aaggacaccagagactcaatttggagctg

SEQ ID NO: 7 - DNA, 21 bp, Artificial Sequence
  Primer
catcaatttggagcagttggg

SEQ ID NO: 8 - DNA, 18 bp, Artificial Sequence
  Primer
cgagggagtcgagtggca

SEQ ID NO: 9 - DNA, 30 bp, Artificial Sequence
  Probe
ttcctagtgatgtcaacaatggcagtaagg

SEQ ID NO: 10 - DNA, 24 bp, Artificial Sequence
  Primer
ggttttctttttcaagctctcaga

SEQ ID NO: 11 - DNA, 18 bp, Artificial Sequence
  Primer
tcgggttcacccttttcg

SEQ ID NO: 12 - DNA, 23 bp, Artificial Sequence
  Probe
aggcatcgtcgccttcaaaggtc

SEQ ID NO: 13 - DNA, 23 bp, Artificial Sequence
  Primer
caataaatgttcgccatctgaca

SEQ ID NO: 14 - DNA, 23 bp, Artificial Sequence
  Primer
aaagctgcgcaaaaactttagtc

SEQ ID NO: 15 - DNA, 16 bp, Artificial Sequence
  Probe
catcaaagaaatcttg

SEQ ID NO: 16 - DNA, 19 bp, Artificial Sequence
  Primer
ggtggtcgccaattagcat

SEQ ID NO: 17 - DNA, 24 bp, Artificial Sequence
  Primer
atacaagccctcaaatctttcaga

SEQ ID NO: 18 - DNA, 17 bp, Artificial Sequence
  Probe
ccattcaagattctttg

(19) United States
(12) Patent Application Publication
(10) Pub. No.: US 2023/0301255 A1
(43) Pub. Date: Sep. 28, 2023

(54) WIDE CROSS BREEDING

(71) Applicant: Syngenta Crop Protection AG, Basel (CH)

(72) Inventors: Qingli Liu, Research Triangle Park, NC (US); Craig Joseph Schuller, Research Triangle Park, NC (US); John Luther Dawson, Research Triangle Park, NC (US); Timothy Joseph Kelliher, Research Triangle Park, NC (US)

(73) Assignee: Syngenta Crop Protection AG, Basel (CH)

(21) Appl. No.: 18/041,274

(22) PCT Filed: Aug. 4, 2021
(86) PCT No.: PCT/US2021/044428

§ 371 Date: Feb. 10, 2023

Related U.S. Application Data
(60) Provisional application No. 63/064,511, filed on Aug. 12, 2020.

(51) Int. Cl. A01H 1/02; A01H 5/10; A01H 6/54

(52) U.S. Cl. CPC A01H 1/026; A01H 1/021; A01H 5/10; A01H 6/542

(57) ABSTRACT

Genes found in wild species can provide beneficial traits to domestic crops. For example, wild Glycine species can provide resistance to a broad spectrum of soy rust populations if introgressed into soybean. Previously, successful introgression required wide cross pollinations, embryo rescue, and regeneration of F1 shoots. Furthermore, chromosome doubling, which can be achieved by chemical treatment of embryos, was required in order to obtain fertile amphidiploids (F1Ds). A backcross to soybean was then required in order to obtain BC1F1 genetics. Here we describe improved procedures for interspecific crosses between soybean and Glycine tomentella. These procedures offer a more efficient route to BC1F1 genetics by bypassing the amphidiploid generation. These principles can be applied to additional domestic species and their wild relatives such as Brassica.

### FIELD OF THE INVENTION

This invention relates to the field of plant breeding. More specifically, this invention relates to interspecific breeding (sometimes called “widecrossing”) between plants not normally capable of cross-fertilization. The invention is also related to doubling a domestic genome to make it more compatible and efficient for crossing with a wild genome.

### SEQUENCE LISTING

This application is accompanied by a sequence listing entitled 82180PCT_ST25.txt, created Aug. 3, 2021, which is approximately 3 kilobytes in size. This sequence listing is incorporated herein by reference in its entirety. This sequence listing is submitted herewith via EFS-Web, and is in compliance with 37 C.F.R. § 1.824(a)(2)-(6) and (b).

### BACKGROUND

Soybean is one of the most important agricultural crops in the world. It is economically vital as it serves as a major source for numerous areas such as food, protein, oil, and other soy products. Glycine contains 2 subgenera, consisting of perennials (subgenus Glycine) and annuals (subgenus Soja). There are more than 26 perennial species that are widely different. Typically, much of soybean breeding to improve cultivars is done with Soja; however, the perennial species have useful agronomic traits of their own that have not been wholly explored. These traits would be useful if bred into cultivated soybean and the use of perennial Glycine would greatly expand the genetic diversity; however, wide hybridization between annuals and perennials is difficult and inefficient by established methods. A wild perennial Glycine species can have an entirely different set and number of chromosomes than the domestic crop plant we call domestic soybean. By way of example and not limitation, wild G. tomentella has 39 distinct chromosomes, while domesticated G. max has 20. Current practice is to produce an infertile hybrid of G. max and G. tomentella. An already extremely inefficient process (low numbers of infertile hybrids are produced), it is further exacerbated by the chromosome doubling process—i.e., the infrequently-obtained infertile hybrid must have its chromosomes doubled by a chemical agent (typically colchicine) to make it fertile, known as a F1D or amphidiploid plant, before it can be bred with G. max. Few survive this process.

There are numerous pathogens that threaten soybean production, e.g. Asian soybean rust (“ASR”) and soybean cyst nematode (“SCN”). Reports state that G. tomentella lines have been found to carry resistance to ASR as well as SCN. See U.S. Pat. No. 7,842,850 (filed May 4, 2006), incorporated herein by reference in its entirety. As an example, soybean rust is caused by two species, Phakopsora pachyrhizi and Phakopsora meibomiae. ASR, caused by P. pachyrhizi, is one of the most damaging diseases affecting legume crops. This aggressive pathogen originated in eastern Asia and was first detected in the continental United States in 2004. It is a parasite that destroys a plant's leaves. P. pachyrhizi can infect more than 95 species, mostly legumes; alternative hosts serve as a reservoir for inoculum build up. Pathogen infection is quick as the spores can infect directly without need for a wound or opening. If temperature and moisture conditions are optimal, infection can occur within 6 hours. Infected plant leaves develop water-soaked spots that progress to reddish brown or tan lesions. The infected foliage turns bronze/yellow and premature defoliation can occur as a result, ultimately affecting the number of pods and seed weight. The spores are spread aerially and under most optimal conditions, a plant can go from first signs of infection to severe defoliation in 1-2 weeks.

Yield losses as high as 80% have been reported due to ASR (See, Kawashima et al. (2016) Nat. Biotechnol. 34:661-65). There are several main control measures utilized for ASR: crop monitoring, chemical fungicides, breeding resistant soybean cultivars, and specific cultivation practices. Incidence of plant diseases can be controlled by agronomic practices that include conventional breeding techniques, crop rotation, and use of synthetic agrochemicals. Conventional breeding methods, however, are time-consuming and require continuous effort to maintain disease resistance as plant pathogens evolve. See, Grover and Gowthaman (2003) Curr. Sci. 84:330-340.

Further, there are many other biotic stresses, fungi as well as bacteria (e.g., bacterial pustule), viruses (e.g., soybean mosaic virus), and nematodes (e.g., soybean cyst nematode “SCN”), that cause issues for soybean production. Improvements are also needed for abiotic stresses (e.g., drought tolerance) and various agronomic traits (e.g., breeding for better yield as well as protein and oil content). To aid in addressing these issues, as well as others, breeding annual Glycine with wild perennial Glycine can be a useful tool. Wild perennial Glycine encompasses a wide range of genomic, cytological, and morphological diversity that can provide a useful source of germplasm.

The difficulty of breeding useful traits into plants is not a problem present only in soybean. Another example of such issues in breeding domestic and wild species includes Brassica species. Typically, a sub-species within a species of Brassica are sexually compatible. However, different species of Brassica do not always exhibit the same compatibility. As an example, wild species B. rapa has 10 chromosomes while domestic species B. oleracea has 9 chromosomes. This chromosomal difference makes the two sexually incompatible. As with soybean, this incompatibility makes it very difficult to transfer a trait from one Brassica species to another.

Considering the significant impact of plant pathogens, pests, and other stresses on the yield and quality of soybean and other crops, other methods are needed for control. Due to the difficulty of breeding annual and perennial Glycine, additional methods are needed for wide hybridization between the two. This invention provides a solution to hybridize the species with fertile plants as a result.

### SUMMARY

Here, we provide methods for efficiently producing fertile hybrids between domestic soy and wild perennial Glycine through use of tetraploid soybean. These methods produce fertile hybrids that can be further backcrossed with soybean to move desirable genes and traits from the wild Glycine into the domestic soybean. This creates a domestic soybean with desirable wild type traits without the need for artificial genetic modification or gene editing.

Also provided is a method of producing at least one hybrid progeny between a wild Glycine species (e.g., G. tomentella) and a domestic soy (i.e., G. max). The hybrid plants produced by this method are generated by crossing doubled domestic soybean with wild Glycine. The doubled domestic soybean is generated first by applying an anti-microtubule agent (e.g., colchicine). This doubled soybean is then crossed with a wild Glycine, and an auxin is optionally applied to obtain at least one fertile hybrid progeny. The F1 hybrid progeny contains 2n^{D} domestic soy chromosomes and 1n^{W} wild Glycine chromosomes. This hybrid progeny can be crossed to the soy plant (yielding a BC1 plant) as well as then crossing the F1 hybrid plant with the wild Glycine plant. In the method, the doubled soy plant and the Glycine tomentella can both either serve as the male or the female plant during crossing. FIG. 1 exemplifies the current invention with reference to tetraploid Glycine species, specifically G. tomentella. This process can be used with other annual species, such as G. soja, and other wild perennial species. Furthermore, the process can be utilized for domestic Brassica species. For example, the process can be carried out using B. oleracea and B. rapa.

This process is an improved method to produce domestic soybean lines containing traits, alleles, or phenotypes from the wild Glycine species in which the domestic soybean originally lacked. An example of such a trait, allele, or phenotype is increased resistance to a pathogen like soybean rust. The methods include the introgression of the trait, allele, or phenotype from the wild Glycine into the domestic soy plant.

### BRIEF DESCRIPTION OF THE SEQUENCES IN THE SEQUENCE LISTING

SEQ ID NOS: 1-2 are primers used in TaqMan assay ID 3289.

SEQ ID NO: 3 is the probe used in TaqMan assay ID 3289.

SEQ ID NOS: 4-5 are primers used in TaqMan assay ID 3316.

SEQ ID NO: 6 is the probe used in TaqMan assay ID 3316.

SEQ ID NOS: 7-8 are primers used in TaqMan assay ID 3434.

SEQ ID NO: 9 is the probe used in TaqMan assay ID 3434.

SEQ ID NOS: 10-11 are primers used in TaqMan assay ID 3435.

SEQ ID NO: 12 is the probe used in TaqMan assay ID 3435.

SEQ ID NOS: 13-14 are primers used in TaqMan assay ID 3537.

SEQ ID NO: 15 is the probe used in TaqMan assay ID 3537.

SEQ ID NOS: 16-17 are primers used in TaqMan assay ID 3538.

SEQ ID NO: 18 is the probe used in TaqMan assay ID 3538.

### BRIEF DESCRIPTION OF THE FIGURES

FIG. 1 is a diagram showcasing the differences between two methods: traditionally introgressing wild Glycine genes into domestic soy and the more efficient method using doubled soy described herein. Once a doubled soy is acquired, the current method avoids the most rate limiting step of the standard process: the colchicine treatment. The method skips the first generation, which is the most time consuming, taking anywhere from 4 to 12 months for completion. There is a reduced need for embryo rescue protocols and the time to mature seeds is quicker. FIG. 1 exemplifies the current method with reference to tetraploid Glycine species, specifically G. tomentella. This process can be used with other annual species, such as G. soja, and other wild perennial species. Furthermore, the process can be utilized for domestic Brassica species. For example, the process can be carried out using B. oleracea and B. rapa.

FIG. 2 displays a confocal image of the spread of 79 F1 chromosomes using chromosome counterstaining DAPI (4′, 6-diamidino-2-phenylindole) at 405 nm excitation. The root material is from a F1 plant from a doubled soy (4n=80) x Glycine tomentella (2n=78) cross.

FIG. 3 displays a confocal image of the labeled centromeres of 40 soy chromosomes with labeled soy centromeres using Alexa Fluor® 488 at 488 nm excitation. The root material is from a F1 plant from a doubled soy (4n=80) x Glycine tomentella (2n=78) cross.

FIG. 4 displays the merged confocal images of the spread of both the 40 soy chromosomes labeled with centromeres and the 39 G. tomentella chromosomes without centromere labeling. The root material is from a F1 plant from a doubled soy (4n=80) x Glycine tomentella (2n=78) cross (n=40+39).

### DEFINITIONS

As used herein, “wild Glycine species” refers to any perennial or annual Glycine species that has not been domesticated. As used herein, “wild perennial Glycine species” refers to any of the more than 26 wild perennial Glycine species with varying genomes (e.g., 2n=38, 40, 78, 80). An example species is Glycine tomentella. As used herein, “n^{W}” refers to the number of distinct chromosomes found in a wild perennial Glycine species. By way of example, and not limitation, Glycine tomentella typically has two copies of 39 distinct chromosomes (comprising a D genome and an E genome), therefore, its n^{W} is 39, while its 2n^{W} is 78. It follows that Glycine tomentella has a genome of DDEE. The term “wild annual Glycine species” refers to, for example, Glycine soja.

As used herein, “domestic annual Glycine species” includes the predominant domesticated Glycine species, Glycine max. Glycine max typically has two copies of 20 distinct chromosomes (each from the G genome), therefore, its n^{D} is 20, while its 2n^{D} is 40. As used herein, “diploid soy plant” or “2n^{D}” refers to a Glycine max plant that has two copies of 20 distinct chromosomes, therefore, its n^{D} is 20, while its 2n^{D} is 40. As used herein, “n^{D}” refers to the number of distinct chromosomes found in a domesticated Glycine species. It follows that Glycine max has a genome of GG. This term can also refer to a soy plant comprising ancestral genes introgressed previously while maintaining a GG genome. The terms soy, soybean, diploid soy plant, Glycine max, and domestic annual Glycine species are used interchangeably throughout.

As used herein, “doubled soy plant” or “tetraploid soy plant” or “4n^{D}” refers to a doubled domestic annual Glycine plant. A typical domestic annual Glycine plant has two copies of 20 distinct chromosomes (each from the G genome), therefore, its n^{D} is 20, while its 2n^{D} is 40, with a genome of GG. These terms can also refer to a doubled soy plant comprising ancestral genes introgressed previously while maintaining a GG genome. A “doubled soy plant” or “tetraploid soy plant” has had its chromosomes doubled through the disruption of spindle fiber formation, typically using a chromosome doubling agent (“CDA”), for example, colchicine, resulting in 4n^{D}, where its 4n^{D} is 80 and has a GGGG genome. A “doubled soy plant” or “tetraploid soy plant” may also be referred to as the “recipient genome”.

As used herein, “recipient species” refers to a species, wherein the genome of that species is doubled to allow for more efficient crossing with an alternate species, e.g., a wild species. For example, a recipient species can be a Glycine max (n^{D} is 20 and 2n^{D} is 40) or Brassica oleracea (n^{D} is 9 and 2n^{D} is 18). These examples of species can be crossed with, for example, a G. tomentella (n^{W} is 39 and 2n^{W} is 78) or Brassica rapa (n^{W} is 10 and 2n^{W} is 20) genome, respectively. The “recipient species” can have its chromosomes doubled for efficient crossing with a wild species; for example, a doubled G. max resulting in 4n^{D}=80 or B. oleracea resulting in 4n^{D}=36. In instances where the recipient species genome is represented without a ^{D} superscript or without a ^{W} superscript, i.e., as “n” for haploid genome, “2n” for diploid genome, etc., the notation reflects the possibility that the recipient species may be either a domestic species or a wild species.

As used herein, “donor species” refers to a species wherein the species serves as the donor of chromosomes when crossing with a recipient species. For example, a donor species can be a Glycine tomentella with n^{W}=39. G. tomentella can be crossed with, for example, doubled G. max (the recipient species) with n^{D} is 20 and 2n^{D} is 40. In instances where the donor species genome is represented without a ^{D} superscript or without a ^{W} superscript, i.e., as “n” for haploid genome, “2n” for diploid genome, etc., the notation reflects the possibility that the donor species may be either a domestic species or a wild species.

As used herein, “hybrid” refers to offspring produced by crossing two genetically dissimilar parent plants.

As used herein, “hybrid progeny” refers to the offspring produced from the cross between the doubled domestic Glycine and the wild Glycine species.

As used herein, “auxin” refers to plant hormones that aid in the elongation of cells ultimately regulating plant growth. An auxin used for this invention can include, but is not limited to, natural or synthetic auxins, such as dicamba (3,6-dichloro-20methoxybenzoic acid), IAA (indole-3-acetic acid), NAA (1-Naphthaleneacetic acid), and 2,4-Dichlorophenoxyacetic acid (2,4-D)

As used herein, chromosome doubling agent (“CDA”) or “anti-microtubule agent” refer to a compound, such as but not limited to colchicine, trifluralin, pronamide, amiprophos-methyl (APM), dithiopyr, carbetamide, chlorthal dimethyl, isopropalin, nitralin, and nitrous oxide, used to interfere with spindle fiber formation. Anti-microtubule agents should be understood to include any protein, peptide, chemical, or other molecule that impairs the function of microtubules, for example, through prevention of tubulin polymerization. Disruption in microtubule formation ultimately leads to inhibition of chromosomal migration, resulting in a cell with a doubled chromosome number.

As used herein, “ploidy” refers to the number of sets of chromosomes in a cell or cells of an organism.

As used herein, “genotype” refers to the genetic make-up of an organism.

As used herein, “desired trait, allele, or phenotype” refers to a characteristic of interest in the wild Glycine species that is desired in the domestic Glycine species. Such a “trait, allele, or phenotype” can include resistance to Asian soy rust, soybean cyst nematode, bacterial pustule, charcoal rot, root rot, and stem canker. Alternatively, such a trait, allele, or phenotype can include improved yield, protein content, oil content, drought tolerance, and flowering times.

As used herein, “introgressed” refers to the introduction of a trait, allele, or phenotype from the genome of one plant, i.e. wild one plant, i.e. wild Glycine, into the genome of another plant, i.e. domestic Glycine, that lacks such trait, allele, or phenotype.

As used herein, “chromosome” refers to, as recognized in the art, the self-replicating genetic structure in the cellular nucleus containing the cellular DNA and bearing the linear array of genes.

As used herein, “self” or “selfing” refers to the production of seed by self-fertilization or self-pollination, i.e. pollen and ovule are from the same plant.

As used here, “F1D” or “amphidiploid” refers to an interspecific hybrid with one complete diploid set of chromosomes derived from each parent species.

### DETAILED DESCRIPTION

We provide a method of producing at least one hybrid progeny between a wild perennial Glycine species and a domestic annual Glycine species. In one embodiment, the method comprises a) obtaining a doubled domestic annual Glycine plant, b) crossing the doubled soy plant with a wild perennial Glycine species plant having 2n^{W} chromosomes, c) applying an auxin, and d) obtaining at least one hybrid progeny therefrom, wherein the hybrid progeny comprises 2n^{D} domestic Glycine and 1n^{W} wild Glycine chromosomes. In one aspect, the method comprises crossing the at least one hybrid progeny to a diploid soy plant to obtain a BC1F1 hybrid plant. In a separate embodiment, the method comprises crossing the F1 hybrid plant with the wild Glycine plant. The at least one hybrid progeny of the method is fertile.

In another embodiment, the doubled soy plant is obtained by using an anti-microtubule agent, wherein the agent is selected from the group consisting of colchicine, trifluralin, and pronamide. The doubled soy plant or plant cell is a Glycine max plant or plant cell, wherein the Glycine max plant or plant cell has a ploidy of 2n^{D}, where n^{D} is the number of distinct domestic chromosomes and where n^{D} is 20. In one aspect, the doubled soy plant has a ploidy of 4n^{D}, where n^{D} is the number of distinct domestic chromosomes, and where n^{D} is 20. The doubled soy plant has a genotype of GGGG and can serve as either the male or the female parent. In one embodiment, the wild Glycine species is a Glycine tomentella. In one aspect, the Glycine tomentella has a ploidy of 2n^{W}, where n^{W} is the number of distinct wild perennial chromosomes, and where n^{W} is 39. The Glycine tomentella has a genotype of DDEE and can serve as either the male or the female parent. The at least one hybrid progeny has a genotype of GGDE.

In one embodiment, the wild Glycine species comprises a desired trait, allele, or phenotype. In one aspect, the desired trait, allele, or phenotype of confers increased resistance to Asian soy rust, soybean cyst nematode, bacterial pustule, charcoal rot, root rot, stem canker, or other soy pathogen. Alternatively, such a trait, allele, or phenotype can include improved yield, protein content, oil content, drought tolerance, and flowering times. Another embodiment relates to a domestic soy plant comprising the trait, allele, or phenotype from the wild Glycine species, wherein the trait, allele, or phenotype is introgressed by the method previously described. Yet another embodiment relates to a hybrid plant produced by the method, wherein the hybrid plant has a genotype of GGDE.

In another embodiment, provided is a method of producing at least one hybrid progeny between a wild plant species and a domestic plant species. Said method comprises a) obtaining a doubled domestic plant having a domestic recipient genome of 4n^{D} chromosomes, b) crossing the doubled domestic plant with a wild species plant having 2n^{W} chromosomes, c) applying an auxin, and d) obtaining at least one hybrid progeny therefrom, wherein the hybrid progeny comprises 2n^{D} domestic plant chromosomes and 1n^{W} wild plant chromosomes. The doubled recipient genome of the method is selected from the group of Glycine, Brassica, Cucurbits, Helianthus, Solanaceae, and Petunia. In one aspect, the method comprises crossing the at least one hybrid progeny to a domestic plant species to obtain a BC1F1 plant. In another aspect, the method further comprises crossing the BC1F1 plant with the wild species plant. The at least one hybrid progeny of the method is fertile.

In another embodiment, provided is a method of producing at least one hybrid progeny between a perennial species and an annual species. Said method comprises a) obtaining a doubled annual plant having a recipient genome of 4n chromosomes, b) crossing the doubled annual plant with a perennial species plant having 2n chromosomes, c) applying an auxin, and d) obtaining at least one hybrid progeny therefrom, wherein the hybrid progeny comprises 2n annual plant chromosomes and 1n perennial plant chromosomes. Yet in another embodiment, provided is a method of producing at least one hybrid progeny between a donor plant species and a recipient plant species. Said method comprises a) obtaining a doubled recipient plant having a genome of 4n chromosomes, b) crossing the doubled recipient plant with a donor plant species having 2n chromosomes, c) applying an auxin, and d) obtaining at least one hybrid progeny therefrom, wherein the hybrid progeny comprises 2n recipient plant chromosomes and 1n donor plant chromosomes.

### EXAMPLES

The applicants evaluated the prior art method of standard introgression. In these practices, Applicants obtained doubled F1 plants (F1D) but were unable to successfully cross the F1Ds with diploid soy to yield BC1s. These results reiterate the difficulty and low efficiency of the prior art method and the need for more efficient methods such as the one described in the following examples.

### Example 1. Method for Obtaining Doubled Soy

Doubled soy lines (tetraploid soy) were generated from two elite lines commonly used in wide crosses, herein referred to as Female 1 and Female 2; these Glycine max lines have 40 chromosomes (G₁ G₁ genome). Immature soybean embryos in tissue culture medium were treated with approximately 0.25-1.0mg/ml colchicine for 3-4 days at 25° C. Regenerated plants were transferred to soil, and leaf samples were taken for ploidy analysis to confirm chromosome doubling. Tetraploid plants were allowed to self, and ploidy analysis was performed on embryos to confirm doubling. An unlimited seed supply was produced by allowing the tetraploid soy to self.

### Example 2. Method of Crossing the Doubled Soy with Wild Glycine

When tetraploid soybean was used as the female parent, soybean flower buds prior to anthesis were prepared by gently removing sepals and petals to expose the mature stigma. Pollen from freshly-opened flowers of G. tomentella (2n=78) was obtained by gently removing the petals to expose the mature anthers and dusting the pollen onto the soybean stigma. When the doubled soybean was used as the male parent, the previously described process was reversed. The wild perennial Glycine used are listed in Table 1. Results from the doubled soy x Glycine crosses can be found in Table 2.

TABLE 1
Wild perennial Glycine lines crossed with tetraploid soy

| Abbreviations | Isozyme Group | Line | 2n Chromosome # |
|---|---|---|---|
| M1 | T1 | PI 441001 | 78 |
| M4 | T5 | PI 509501 | 78 |
| M5 | T1 | PI 483224 | 78 |
| M14 | T1 | PI 499939 | 78 |
| M15 | D3 | PI 505267 | 40 |

TABLE 2
Totals for each doubled soy × perennial Glycine combination

| | | | | | | Shoots/ |
|---|---|---|---|---|---|---|
| | | | Embryos/ | | Pollinations/ | Embryo |
| Cross | Pollinations | Embryos | Cross | Shoots | Shoot | (%) |
| Female 1 × M1 | 20 | 10 | 0.50 | 1 | 20 | 10 |
| Female 2 × M1 | 12 | 8 | 0.67 | 2 | 6 | 25 |
| Female 1 × M14 | 604 | 293 | 0.49 | 23 | 26.3 | 7.8 |
| Female 2 × M14 | 78 | 23 | 0.29 | 2 | 39 | 8.7 |
| Female 1 × M4 | 46 | 15 | 0.33 | 1 | 46 | 6.7 |
| Female 1 × M5/M14 | 61 | 22 | 0.36 | 2 | 30.5 | 9.1 |
| Female 1 × M15 | 22 | 4 | 0.18 | 1 | 22 | 25 |
| Female 2 × M15 | 28 | 2 | 0.07 | 0 | N/A | N/A |
| Female 1 × M5 | 9 | 4 | 0.44 | 0 | N/A | N/A |

### Example 3. Auxin Application

Dicamba, a synthetic auxin herbicide, was sprayed on tetraploid x Glycine crosses to produce pod and embryo formation. As seen in Table 3, dicamba was sprayed at a 3 to 20 mg/L concentration. A spray bottle or atomizer was used to achieve good saturation of the pollinated gynoecia and the node to which it was attached. Also shown in Table 3, various application days were evaluated (i.e. 1-5 days of spraying).

TABLE 3
A table showing the various auxin (dicamba) treatments for all crosses.

| Dicamba | # of Application | # of | # of | Embryos/ | |
|---|---|---|---|---|---|
| Treatment* | Days | Pollinations | Embryos | Cross | Shoots |
| BC 3D | 5 Days | 34 | 15 | 0.44 | 3 |
| BC 5D | 1 Day | 4 | 0 | 0.00 | 0 |
| BC 5D | 2 Days | 332 | 139 | 0.42 | 10 |
| BC 5D | 3 Days | 249 | 110 | 0.44 | 10 |
| BC 7D | 3 Days | 86 | 32 | 0.37 | 3 |
| BC 10D | 1 Day | 34 | 9 | 0.26 | 0 |
| BC 10D | 2 Days | 93 | 53 | 0.57 | 4 |
| BC 10D | 3 Days | 38 | 21 | 0.55 | 2 |
| 20D | 2 Days | 9 | 2 | 0.22 | 0 |

*BC contains 100 mg/L GA3 + 5 mg/L kinetin; D = mg/L dicamba
*BC 3D contains 3 mg/L dicamba, BC 5D contains 5 mg/L dicamba, etc.
*20D contains 20 mg/L dicamba onlv

### Example 4. Backcrossing

Once the F1 was obtained by crossing the doubled soy with the wild Glycine, the F1 was then backcrossed with domestic soy to obtain BC1F1. A second backcross with domestic soy was done to arrive at BC2F1 progeny. With the current method, fewer embryo rescue steps are needed to arrive at mature seed. As shown in Table 4, successful generation of BC2F1 progeny was accomplished using the new, more efficient tetraploid soy parent introgression method.

TABLE 4
Results from backcrossing the F1 hybrid
with domestic soy for two generations

| | | # of BC1F1 | # of BC2F1 |
|---|---|---|---|
| Cross | F1 Shoot ID # | Progeny* | Progeny** |
| Female 1 x M1 | 1 | 2 | 9 |
| Female 1 x M14 | 2 | 0 | — |
| Female 1 x M14 | 3 | 1 | 0 |
| Female 1 x M14 | 4 | 0 | — |
| Female 1 x M14 | 5 | 0 | — |
| Female 1 x M14 | 6 | 0 | — |
| Female 1 x M14 | 7 | 10 | 2 |
| Female 1 x M14 | 8 | 0 | — |
| Female 1 x M14 | 9 | 1 | 0 |
| Female 1 x M14 | 10 | 0 | — |
| Female 1 x M14 | 11 | 1 | 0 |
| Female 1 x M14 | 12 | 1 | 0 |
| Female 1 x M14 | 13 | 2 | 12 |
| Female 1 x M14 | 14 | 0 | — |
| Female 1 x M14 | 15 | 1 | 0 |
| Female 1 x M14 | 16 | 0 | — |
| Female 1 x M14 | 17 | 0 | — |
| Female 1 x M14 | 18 | 0 | — |
| Female 1 x M14 | 19 | 6 *** | — |
| Female 1 x M14 | 20 | 4 | 21 |
| Female 1 x M14 | 21 | 3 | 0 |
| Female 1 x M14 | 22 | 0 | — |
| Female 1 x M14 | 23 | 0 | — |
| Female 1 x M14 | 24 | 0 | — |
| Female 1 x M5/M14 | 25 | 0 | — |
| Female 1 x M5/M14 | 26 | 0 | — |
| Female 2 x M14 | 27 | 0 | — |
| Female 2 x M14 | 28 | 0 | — |

*Rooted shoots in soil except as noted
**Mature seeds
***Small mature seeds; germination not attempted

### Example 5. F1 Plants of the Current Method with Increased Resistance to Soybean Rust

F1 plants produced through the method of introgression using tetraploid soybean were evaluated for resistance to soybean rust. Results for some of these plants are shown in Table 5. Three soy rust strains were used to evaluate resistance of each F1 plant and a soybean control. “RB” represents “reddish brown” lesions while the number associated with the acronym represents a value related to the plant's resistance to the rust strain. The lower the value, the more resistant to the strain it is. “SP” represents “sporulation”. For example, “no-sp” translates to “no sporulation” on the plant while “sp-medium” and “sp-little” translates to a subjective observation of little to medium sporulation of the pathogen on the plant. The bottom row displays the soybean control data. Here, “tan” represents a susceptible reaction with the number associated representing the plant's level of susceptibility. The higher the value, the more susceptible the plant is, with 5 being the most susceptible. Compared to the control, the F1 plants show increased resistance to all three strains of the rust pathogen.

TABLE 5
Resistance to soybean rust for F1 plants produced through
the tetraploid introgression method

| Plant ID | Soy rust strain 1 | Soy rust strain 2 | Soy rust strain 3 |
|---|---|---|---|
| F1-1 | RB4 sp-medium | RB2 no-sp | RB2 no-sp |
| F1-2 | RB4 sp-little | RB2 no-sp | RB2 no-sp |
| F1-3 | RB4 sp-little | RB2 no-sp | RB2 no-sp |
| F1-4 | RB4 sp-little | RB2 no-sp | RB2 no-sp |
| Soybean (control) | Tan5-sp | Tan4-sp | Tan4-sp |

RB = reddish brown lesions; sp = sporulation; # = a scale (1-5) for resistance to
susceptibility with the lower values representing more resistant and higher values
representing more susceptible to the rust pathogen

### Example 6. Confirmation of True Hybrids

To verify the crosses produced true hybrids, TaqMan assays were completed.

TABLE 6
Taqman data for all crosses to confirm hybrids

| | F1 | TaqMan | TaqMan | TaqMan | TaqMan | TaqMan | TaqMan |
|---|---|---|---|---|---|---|---|
| | Shoot | 3289 | 3316 | 3434 | 3435 | 3537 | 3538 |
| | ID | Copy# | Copy# | Copy# | Copy# | Copy# | Copy# |
| Cross | # | level | level | level | level | level | level |
| Female 1 | Control | 0 | 0 | 0 | 0 | 0 | 0 |
| Female 1 × M1 | 1 | 1 | 1 | 1 | 1 | 0 or 1 | 1 or 2 |
| Female 1 × M14 | 2 | 2 | 1 | 1 | 1 or 2 | 1 | 1 |
| Female 1 × M14 | 3 | 2 | 1 | 1 | 1 | 1 | 1 |
| Female 1 × M14 | 4 | 2 | 1 | 1 | 1 | 1 | 1 |
| Female 1 × M14 | 5 | 2 | 1 | 1 | 1 | 1 | 1 |
| Female 1 × M14 | 6 | 2 | 1 | 1 | 1 | 1 | 1 |
| Female 1 × M14 | 7 | 2 | 1 | 1 | 1 or 2 | 1 | 1 |
| Female 1 × M14 | 8 | 2 | 1 | 1 | 1 or 2 | 1 | 1 |
| Female 1 × M14 | 9 | 2 | 1 | 1 | 1 or 2 | 1 | 1 |
| Female 1 × M14 | 10 | 2 | 1 | 1 | 1 or 2 | 1 | 1 |
| Female 1 × M14 | 11 | 2 | 1 | 1 | 1 | 1 | 1 |
| Female 1 × M14 | 12 | 2 | 1 | 1 | 1 or 2 | 1 | 1 |
| Female 1 × M14 | 13 | 2 | 1 | 1 | 1 | 1 | 1 |
| Female 1 × M14 | 14 | 2 | 1 | 0 or 1 | 1 | 1 | 1 |
| Female 1 × M14 | 15 | 2 | 1 | 1 | 1 | 1 | 1 |
| Female 1 × M14 | 16 | 2 | 1 | 1 | 2 | N/A | N/A |
| Female 1 × M14 | 17 | 2 | 1 | 0 or 1 | 1 | 1 | 1 |
| Female 1 × M14 | 18 | 2 | 1 | 0 or 1 | 1 | 1 | 1 |
| Female 1 × M14 | 19 | 1 or 2 | 1 | 0 or 1 | 1 | 1 | 1 |
| Female 1 × M14 | 20 | 2 | 1 | 1 | 1 | 1 | 1 |
| Female 1 × M14 | 21 | 2 | 1 | 1 | 2 | N/A | N/A |
| Female 1 × M14 | 22 | 1 | 1 | 1 | 1 | 1 | 1 |
| Female 1 × M14 | 23 | 2 | 1 | 1 | 2 | N/A | N/A |
| Female 1 × M14 | 24 | 1 | 1 | 1 | 1 | 1 | 1 |
| Female 1 × | 25 | 1 | 1 | 0 or 1 | 0 or 1 | 1 | 1 |
| M5/M14 | | | | | | | |
| Female 1 × | 26 | 1 | 1 | 0 or 1 | 1 | 1 | 1 |
| M5/M14 | | | | | | | |
| Female 2 × M14 | 27 | 2 | 1 | 0 or 1 | 1 | 1 | 1 |
| Female 2 × M14 | 28 | 2 | 1 | 0 | 1 | 1 | 1 |
| Female 1 × M4 | 29 | 2 | 1 | 1 | 1 | 1 | 0 |
| Female 1 × M15 | 30 | 0 | 1 | 1 | 0 | 1 | 0 |

### Example 7. Root Chromosome Spread Staining and Imaging

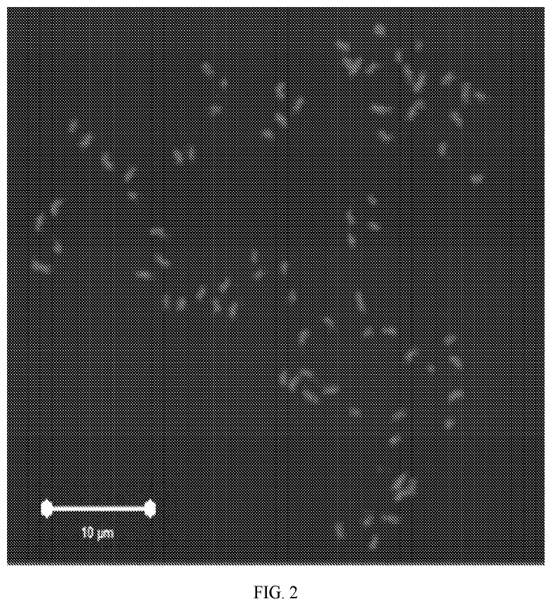

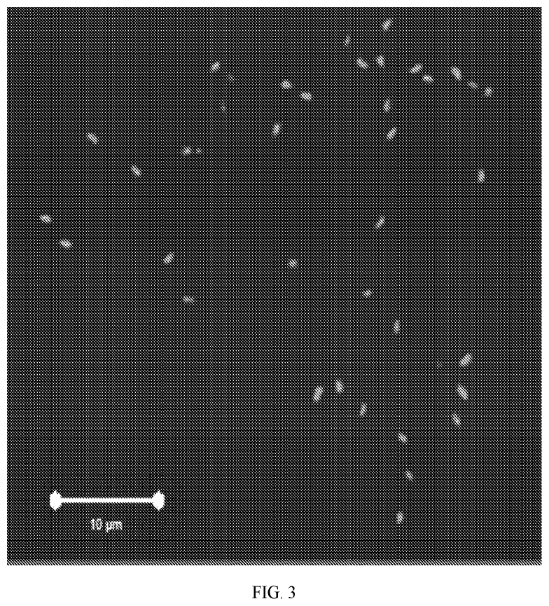

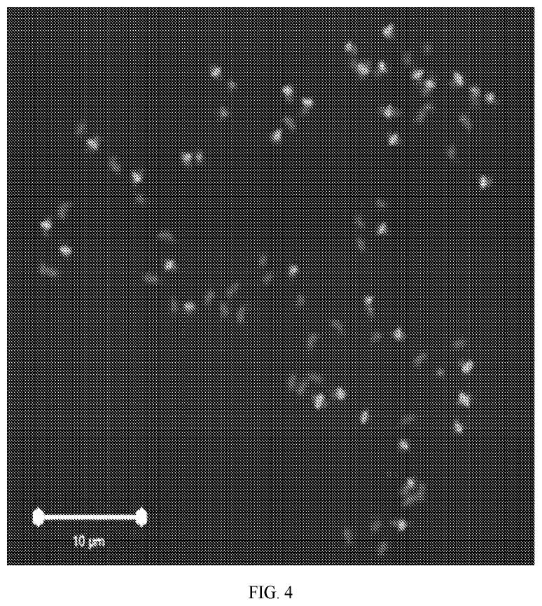

Fluorescence in situ hybridization (FISH) technology was used to distinguish between soy chromosomes and wild Glycine chromosomes. The root material in FIGS. 2, 3, and 4 is from a F1 plant (2n^{D}1nW=40+39) from a doubled soy (4n^{D}=80) x G. tomentella, (2n^{W}=78) cross. Soy centromeres were identified as cytological markers and used for FISH. The soy centromeres were labeled with Alexa Fluor® 488 by nick translation, which does not hybridize to G. tomentella centromeres, only to soybean centromeres. Soy chromosomes were visualized using chromosome counterstain DAPI (4′, 6-diamidino-2-phenylindole). Using FISH, the 40 soy chromosomes were distinguished from the 39 G. tomentella chromosomes in the F1 plant.

For FISH, root materials were first arrested in 0.05% 8-hydroxyquinoline for 5 hours and fixed in 90% acetic acid. The root tips (meristem region) were cut and briefly digested with 0.5% pectolyase and 1% cellulase to remove cell walls. Soy centromeric repeats, CentGm-1 and CentGm-2, were amplified by PCR from soybean genomic DNA according to the literature (See Gill et al., Plant Physiol. 2009 Vol 151, p1167-1174), and centromere probes were labeled with fluorescent dye by nick translation. Chromosomes were spread and hybridized with the soy centromere probes and chromosomes were counterstained with DAPI followed by examination and imaging under a confocal microscope (Zeiss 710).

### Example 8. Efficiency Gains with Current Procedures

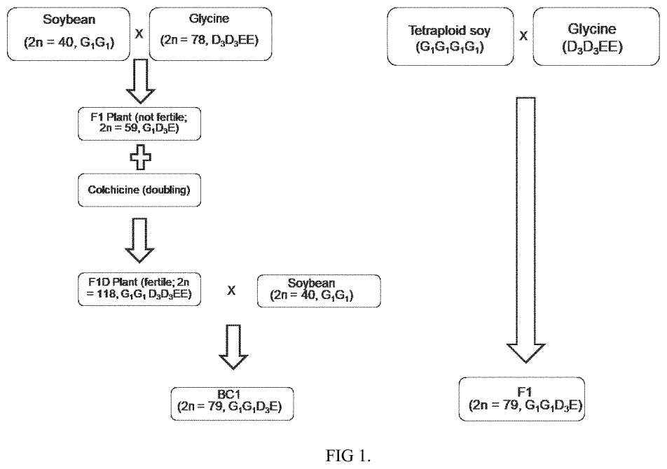

As seen in Table 7 and FIG. 1, there are great improvements in the current methodology compared to the standard procedure of introgression. Table 7 displays the results of crossing two different Glycine lines per the standard, starting with diploid soy, as well as crossing the Glycine lines with tetraploid soy. Not only is the current method quicker, by eliminating steps, but it is more efficient, yielding a 280-647-fold efficiency gain.

TABLE 7
This table demonstrates the improvements in efficiency utilizing the current
procedures over standard methods of introgression.

| | | # | # | | # of | Avg Days | Fold |
|---|---|---|---|---|---|---|---|
| Glycine | | Pollinations | Pollinations | Total # | GGDE | to GGDE | Efficiency |
| Line | Route | for F1Ds | for GGDE¹ | Pollinations | Shoots | Shoot² | Gain³ |
| M1 | Standard | 5957 | 1800 | 7757 | 7 | 461 | n/a |
| M1 | Tetra | 0 | 32 | 32 | 3 | 74 | 647 |
| | Soy | | | | | | |
| M14 | Standard | 3150 | approx. 100 | approx. 3250 | 0 | n/a | n/a |
| M14 | Tetra | 0 | 681 | 681 | 25 | 67 | 280 |
| | Soy | | | | | | |

¹GGDE genetics: B1 generation by standard route, F1 generation by tetra soy route
²Number of days from Soy × Glycine wide cross until GGDE shoot in soil
³Compared to M1 Standard; considers total # of pollinations and time to acquire GGDE shoots

### Example 9. Wide Cross Breeding with Brassica

### Chromosome Doubling of Domesticated Brassica Species.

The methods described above for soybean can be applied to Brassica species. First, obtain a doubled domestic Brassica species (for example, B. oleracea) by doubling the chromosomes using a chromosome doubling agent (e.g. colchicine). To do so, treat immature embryos in tissue culture medium with approximately 0.25-1.0 mg/ml colchicine for 3-4 days at 25° C. Next, transfer regenerated plants to soil and take leaf samples for ploidy analysis to confirm doubling. Allow tetraploid plants to self and perform ploidy analysis on embryos to confirm doubling.

Cross the Doubled Domestic Brassica with a Wild Brassica Species.

Next, cross the doubled species with a wild Brassica species (for example, B. rapa). When using tetraploid Brassica as the female parent, gently remove sepals and petals to expose the mature stigma, prior to anthesis, to prepare the Brassica flower buds. Gently remove pollen from freshly-opened flowers of the wild species by gently removing the petals to expose the mature anthers and dusting the pollen onto the Brassica stigma. If the doubled Brassica is used as the male parent, the previously described process can be reversed. This cross will result in an F1 with the BC1F1 genetics of a standard introgression method.

### Apply Auxin

Spray dicamba, a synthetic auxin herbicide, on tetraploid x wild Brassica pollination attempts to produce pod and embryo formation. Dicamba application may range from a 3 to 20 mg/L concentration. To achieve good saturation of the pollinated gynoecia and the node to which it is attached, use a spray bottle or atomizer. Various application days can be evaluated (e.g. 1-5 days of spraying).

### Backcross

Once the F1 is obtained by crossing the doubled Brassica with the wild Brassica, backcross the F1 with domestic Brassica to obtain BC1F1. Complete a second backcross with domestic Brassica to arrive at BC2F1 progeny. With the current method, fewer embryo rescue steps are needed to arrive at mature seed.

1-34. (canceled)

35. A method of producing at least one hybrid progeny between a wild perennial Glycine species and a domestic annual Glycine species, the method comprising:
a. obtaining a doubled domestic annual Glycine plant having a domestic recipient genome of 4n^{D} chromosomes;
b. crossing the doubled soy plant with a wild perennial Glycine species plant having 2n^{W} chromosomes; and
c. applying an auxin;
d. obtaining at least one hybrid progeny therefrom;
e. wherein the hybrid progeny comprises 2n^{D} domestic Glycine and 1n^{W} wild Glycine chromosomes.

36. The method of claim 35, further comprising crossing the at least one hybrid progeny to a diploid soy plant to obtain an BC1F1 soy plant.

37. The method of claim 36, further comprising crossing the BC1F1 soy plant with the wild Glycine plant.

38. The method of claim 35, wherein the at least one hybrid progeny is fertile.

39. The method of claim 35, wherein the doubled soy plant is obtained by an anti-microtubule agent, wherein the agent is selected from the group consisting of colchicine, trifluralin, and pronamide.

40. The method of claim 35, wherein the soy plant or plant cell is a Glycine max plant or plant cell.

41. The method of claim 40, wherein the Glycine max plant or plant cell has a ploidy of 2n^{D}, where n^{D} is the number of distinct domestic chromosomes.

42. The method of claim 41, wherein the Glycine max plant or plant cell has a ploidy of 2n^{D}, where n is 20 distinct domestic chromosomes.

43. The method of claim 35, wherein the doubled soy plant has a ploidy of 4n^{D}, where n^{D} is the number of distinct domestic chromosomes.

44. The method of claim 43, wherein the doubled soy plant has a ploidy of 4n^{D}, where n^{D} is 20 distinct domestic chromosomes.

45. The method of claim 43, wherein the doubled soy plant has a genotype of GGGG.

46. The method of claim 45, wherein the doubled soy plant serves as the female parent.

47. The method of claim 45, wherein the doubled soy plant serves as the male parent

48. The method of claim 35, wherein the wild Glycine species is a Glycine tomentella.

49. The method of claim 48, wherein the Glycine tomentella has a ploidy of 2n^{W}, where n^{W} is the number of distinct wild perennial chromosomes.

50. The method of claim 49, wherein the Glycine tomentella has a ploidy of 2n^{W}, where n^{W} is 39 distinct wild perennial chromosomes.

51. The method of claim 50, wherein the Glycine tomentella has a genotype of DDEE.

52. The method of claim 51, wherein the Glycine tomentella serves as the male parent.

53. The method of claim 51, wherein the Glycine tomentella serves as the female parent.

54. The method of claim 35, wherein the at least one hybrid progeny has a genotype of GGDE.

55. The method of claim 35, wherein the wild Glycine species comprises a desired trait, allele, or phenotype.

56. The method of claim 55, wherein the desired trait, allele, or phenotype comprises resistance to Asian soy rust, soybean cyst nematode, bacterial pustule, charcoal rot, root rot, stem canker, or other soy pathogen.

57. The method of claim 55, wherein the desired trait, allele, or phenotype confers improved yield, protein content, oil content, drought tolerance, or flowering times.

58. A hybrid plant produced by the method of claim 35, wherein the hybrid plant has a genotype of GGDE.

59. A method of producing at least one hybrid progeny between a wild plant species and a domestic plant species, the method comprising:
a. obtaining a doubled domestic plant having a domestic recipient genome of 4n^{D} chromosomes;
b. crossing the doubled domestic plant with a wild species plant having 2n^{W} chromosomes; and
c. applying an auxin;
d. obtaining at least one hybrid progeny therefrom;
e. wherein the hybrid progeny comprises 2n^{D} domestic plant chromosomes and 1n^{W} wild plant chromosomes.

60. The method of claim 59, wherein the doubled recipient recipient genome is selected from the group of Glycine, Brassica, Cucurbits, Helianthus, Solanaceae, and Petunia.

61. The method of claim 59, further comprising crossing the at least one hybrid progeny to a domestic plant species to obtain a BC1F1 plant.

62. The method of claim 61, further comprising crossing the BC1F1 plant with the wild species plant.

63. The method of claim 59, wherein the at least one hybrid progeny is fertile.

64. A method of producing at least one hybrid progeny between a perennial species and an annual species, the method comprising:
a. obtaining a doubled annual plant having a recipient genome of 4n chromosomes;
b. crossing the doubled annual plant with a perennial species plant having 2n chromosomes; and
c. applying an auxin;
d. obtaining at least one hybrid progeny therefrom;
e. wherein the hybrid progeny comprises 2n annual plant chromosomes and 1n perennial plant chromosomes.

65. A method of producing at least one hybrid progeny between a donor plant species and a recipient plant species, the method comprising:
a. obtaining a doubled recipient plant having a genome of 4n chromosomes;
b. crossing the doubled recipient plant with a donor plant species having 2n chromosomes; and
c. applying an auxin;
d. obtaining at least one hybrid progeny therefrom;
e. wherein the hybrid progeny comprises 2n recipient plant chromosomes and 1n donor plant chromosomes.
